(19) United States
(12) Patent Application Publication
(10) Pub. No.: US 2004/0133227 A1
(43) Pub. Date: Jul. 8, 2004

(54) Retractable skin nicker

(76) Inventors: Walter Shang, Salt Lake City, UT (US); Dwight Hibdon, Park City, UT (US); Kelly B. Powers, Salt Lake City, UT (US)

Correspondence Address: MORRISON & FOERSTER, LLP, 555 WEST FIFTH STREET, SUITE 3500, LOS ANGELES, CA, 90013-1024, US

(21) Appl. No.: 10/337,149

(22) Filed: Jan. 6, 2003

(51) Int. Cl. A61B 17/32

(52) U.S. Cl. 606/182

(57) ABSTRACT

A surgical cutting device for producing a skin incision of controlled length and depth at a guidewire skin puncture location. The cutting device is particularly useful for making incisions for the insertion of catheters. In one variation, the cutting device comprises a retractable blade and a channel for positioning the cutting device around a guidewire. The channel may be adapted such that when a guidewire is placed inside the cutting device, the guidewire may exit the channel at a 30 degree angle relative to the base of the cutting device. Methods for using the cutting device are also described.

### FIELD OF THE INVENTION

The present invention relates to a surgical device for creating precise, small skin incisions for the introduction of a percutaneous conduit. In particular, the invention relates to such a device, which is designed for creating a small skin incision of controlled geometry about a puncture created by a guidewire or needle for the purpose of introducing a catheter or other percutaneous conduit.

### BACKGROUND OF THE INVENTION

Many medical procedures require the deployment of a percutaneous device or conduit which penetrates the skin and allows connection of a cavity or organ inside the body with equipment that are externally located. For example, in the facilitation of fluid transport, percutaneous conduits or catheters, are used to access blood vessels for dialysis, pressure monitoring, laboratory diagnosis, drug delivery, nutritional solution delivery, or to drain excess fluid from a wound.

In the placement of catheters, it is often desirable to enlarge the puncture hole created initially by a needle. The enlarged hole allows the introduction of a larger diameter catheter. Generally, an introducer device such as a needle attached to a syringe, is used to insert a catheter in a blood vessel. One common approach is the Seldinger technique. A needle is attached to a syringe and inserted under the patient's skin, the vessel is identified by aspiration using the suction from the syringe. When blood enters the syringe, indicating that the vessel has been found, the syringe body is removed and a guidewire is introduced through the needle lumen and into the interior of the vessel. The needle is then removed, leaving a portion of the guidewire within the vessel and the remainder projecting outwardly beyond the surface of the patient's skin. A catheter or other introducing device may then be inserted over the guidewire using one of several techniques well known to one skilled in the art. In order to introduce a catheter or other devices (e.g. sheath, dilator, introducer), which has a larger diameter, over the guidewire and into the patient's body, an incision often must be made around the guidewire at the point of entry into the patient's body. Typically, the incisions are made with a No. 11 scalpel blade or an equivalent device by cutting outwardly from the wire. Finally, a catheter is inserted along the guidewire through the skin into the deeper tissues.

The skin incision step is particularly problematic since it is difficult to make a cut of the desired width and depth necessary for introduction of the catheter without forming a cut either greater or smaller than desired. Since the guidewire is inserted inside a vessel within soft tissues, the guidewire and the tissue may move easily relative to each other. In addition, the medical practitioner also needs to hold on to the guidewire to maintain the proper position of the guidewire. With the other hand holding on to the scalpel, and having no appropriate anchor point for the knife or the hand, it is generally difficult to make a precise incision with the appropriate length and depth. Furthermore, care must be taken not to sever the guidewire when making the skin incision, since the resulting internal piece of wire might then travel within the vascular system, resulting in various complications.

Because the purpose of the guidewire is to facilitate insertion of catheters or other percutaneous device into the vessel, it is preferable that the guidewire enters the tissue at an appropriate angle. Poor positioning of the guidewire may result in damage to the vessel, puncture of the vessel and/or coagulation inside the vessel. Thus, maintaining the appropriate insertion angle and making an incision with the appropriate depth around the site of guidewire insertion may be critical to successful placement of the catheter.

It is also known that a significant number of nurses and clinicians are uncomfortable with operating a surgical scalpel. The risk of cutting oneself will increase proportionately with the time spend handling sharp cutting devices. Under high stress environments such as an emergency room or at the seen of a car accident, the risk of accidental contact with the blade is further amplified.

The associated risks of accidental cutting after the completion of medical procedures have became a public health problem due to increasing number of patient's carrying blood borne pathogens, such as HIV virus or hepatitis. After the completion of the cutting procedure, the exposed blade (usually containing blood and/or bodily fluids) poses a significant danger to health care professionals. The danger occurs during the use of the scalpel, in cleaning up the operating room or patient's room, or in subsequent disposal of the used scalpel.

While no specific data is available with regard to accidental injury due to scalpels, the risk is at least comparable to the problems associated with needle sticks. The Centers for Disease Control and Prevention (CDC) has reported that about 384,000 needle-stick or similar injuries occur among U.S. hospital health-care workers every year. In addition, in one study, out of 3,978 needle sticks from patients known to be HIV positive, 13 health care workers became infected, roughly 1 out of 300.

Various mechanisms have been devised for cutting and introducing tubing into the human body. Examples of such devices are disclosed in U.S. Pat. No. 2,753,105, issued Jul. 3, 1956 to Werner et al.; U.S. Pat. No. 3,863,339, issued Feb. 4, 1975 to Reaney et al.; U.S. Pat. No. 3,895,411, issued Jul. 22, 1975 to Horak; U.S. Pat. No. 3,906,626, issued Sep. 23, 1975 to Riuli; U.S. Pat. No. 4,414,974, issued Nov. 15, 1983 to Dotson et al.; U.S. Pat. No. 4,438,770, issued Mar. 27, 1984 to Ungey et al.; U.S. Pat. No. 4,601,710, issued Jul. 22, 1986 to Moll; U.S. Pat. No. 4,617,738, issued Oct. 21, 1986 to Kopacz; U.S. Pat. No. 4,633,860, issued Jan. 6, 1987 to Korth et al.; U.S. Pat. No. 4,643,189, issued Feb. 17, 1987 to Mintz; U.S. Pat. No. 4,902,280, issued Feb. 20, 1990 to Lander; U.S. Pat. No. 4,760,848, issued Aug. 2, 1988 to Hasson; U.S. Pat. No. 4,955,887, issued Sep. 11, 1990 to Zirm; U.S. Pat. No. 4,955,890, issued Jun. 18, 1996 to Yamamoto et al.; U.S. Pat. No. 5,066,288, issued Nov. 19, 1991 to Deniga et al.; U.S. Pat. No. 5,139,507, issued Aug. 18, 192 to Dolgin et al.; U.S. Pat. No. 5,186,168, issued Feb. 16, 1993 to Spofford et al.; U.S. Pat. No. 5,208,983, issued May 11, 1993 to Masse; U.S. Pat. No. 5,211,652, issued May 18, 1993 to Derbyshire; U.S. Pat. No. 5,370,654, issued Dec. 6, 1994 to Abidin et al.; U.S. Pat. No. 5,527,333, issued Jun. 18, 1996 to Nikkels et al.; U.S. Pat. No. 5,529,581, issued Jun. 25, 1996 to Cusack; U.S. Pat. No. 5,545,175, issued Aug. 13, 1996 to Abidin et al.; U.S. Pat. No. 5,749,886, issued May 12, 1998 to Abidin et al.; U.S. Pat. No. 5,843,108, issued Dec. 1, 1998 to Sammuels; U.S. Pat. No. 6,221,047, issued Apr. 24, 2001 to Greene et al.; U.S. Pat. No. 6,238,369 B1, issued May 29, 2001 to Burbank et al.; U.S. Pat. No. 6,277,100 B1, issued Aug. 21, 2001 to Raulerson et al.; U.S. Pat. No. 6,402,770 B1, issued Jun. 11, 2002 to Jessen; each of which is incorporated in its entirety by reference herein. Although the above references suggests various cutting mechanisms, they do not teach nor suggest a device with safety features and a channel for providing guidance for appropriate angle of deployment of the device.

Therefore, a device equipped with a retraceable surgical blade, adapted for placement over a guidewire, which may be used as guidance to locate the insertion site, and capable of making a skin nick with predetermined incision angle at the insertion point of the guidewire or introducer needle is desirable. Such a device may significantly improve both the quality and safety of procedures for deployment of a percutaneous device or conduit in patients.

### SUMMARY OF THE INVENTION

Accordingly, one of the objectives of the present invention is to provide a cutting device equipped with a surgical blade for microintroduction. It is another objective of the present invention to provide a cutting device for placement over a guidewire, which may be used as guidance to locate the insertion site. It is yet another objective of the present invention to provide a cutting device that is able to make a skin nick at the insertion point of the introducer needle to enlarge the hole for accommodating various french sizes (e.g., 3.5 to 7.0 fr.) of splittable sheath/dilators. It is a further objective to provide a cutting device with safety features (e.g. retractable blade, blade-locking mechanisms). It is also a further objective to provide a cutting device- that may be easily operated with one-hand. Various other objectives and advantages of the present invention will become apparent to those skilled in the art as more detailed description is set forth below.

One aspect of the present invention provides a cutting device with a retractable blade for making incisions around a guidewire, which is partially inserted into a mammalian body, to enlarge the point of entry. The cutting device may comprise of a housing with a channel so that the housing may be slidably placed over a guidewire. The guidewire may be used as guidance to locate the insertion site by sliding the cutting device on the guidewire towards the insertion location. The cutting device may further comprise a retractable blade adapted for making an incision at the guidewire entry location once the housing is positioned at the cite of insertion. Various retractable blade mechanisms well known to one skilled in the art may be adapted for this cutting device.

The cutting device may have a bottom surface such that when the cutting device is positioned over the guidewire and at the point of insertion, the bottom surface comes into contact with the subjects skin surface. The bottom surface may have a large enough surface area to provide stability for the cutting device when actuating the cutting device. Preferably, the bottom surface has a surface area between about 0.5 square centimeters to about 10 square centimeters. More preferably, the bottom surface has a surface area of about 1 square centimeters to about 7 square centimeters. Even more preferably, the bottom surface has a surface area of about 2 square centimeters to about 6 square centimeters. The bottom surface may be a smooth flat surface or alternatively it may have groves and/or notches. The bottom surface may comprise of a planner area or a virtual plan formed of multiple support points extended from the housing, such that the planner area is parallel to the skin surface when the housing is placed on the skin of the subject. In one variation, the bottom of the housing has an arc, such that when the housing sits on a planner surface, the two end points of the arc come into contact with the planner surface. In this case, the two end points of the arc form the bottom surface of the housing. In another variation, the bottom surface is comprised of plurality of arcs.

In another aspect of the invention, a channel is provided such that when a guidewire is passed through the housing, the guidewire exits the housing at a predefined angle (i.e., the incision angle) from the bottom surface of the housing. The predefined angle may be selected based on the particular application for which the cutting device is designed and should generally be in the range of approximately 10 degrees to 60 degrees. As shown and described herein with respect to using in an application for making incisions around a guidewire, the incision angle should be in the range of approximately 25 degrees to 35 degrees.

In another variation, the predefined angle may be adjustable on the cutting device. The cutting device may have a pivot allowing the position of the channel be adjusted relative to the bottom surface. The cutting device may further have a locking mechanism for securing the bottom surface once the desired angle is selected. For example, the cutting device with a variable angle adjustment mechanism may be preset at 30 degrees. The medical professional using the device may reset the angle to 45 degrees before applying the cutting device, if such angle is desirable.

In one variation of the invention the guidewire channel may be enclosed within the chamber except that the distal end and proximal end of the channel are exposed at opposite sides of the housing. The channel may be straight and allow the guidewire to pass through without bending. Alternatively, the channel may partially bend, such that the guidewire enters the housing at an angle (relative to the bottom surface) that is greater than the angle (between the guidewire and the bottom surface) from which the guidewire exists the housing. In another variation, the channel may have a curvature.

In another aspect of the invention, the channel may be partially open along the channel on at least one side of the channel. In one variation, the housing has a relatively flat and elongated profile. The channel runs from the distal end of the housing to the proximal end of the housing. Along the length of the channel, the channel may be exposed on one side such that a guidewire may be placed inside the channel laterally without the need to slide the housing on to the guidewire through either the distal end or proximal end of the channel.

The housing may further include a locking mechanism such that once the guidewire is laterally slid into the channel the locking mechanism may be activated to prevent the guidewire from sliding out of the channel laterally. This may allow the housing to slide along the guidewire securely without the risk of detachment form the guidwire through the lateral opening. When the user completes the cutting procedure, he may unlock the locking mechanism and remove the housing firm the lateral opening in the guidewire. Alternatively, a flap or other flexible materials may be placed along the chamber on the exposed side of the channel, such that to place the housing over the guidewire laterally, or to remove the housing from the guidewire laterally, would require the user to overcome a resistive force.

In one variation, the retractable blade is a surgical grade stainless steel blade. In an alternative variation, the surgical blade is a No. 11 surgical blade. The surgical blade may be coupled to a elastic member such as a spring. The spring may keep the blade in the chamber when the cutting device is not actuated. The spring may comprise a resilient plastic, an elastic metal, an elastic alloy, an elastic polymer, natural rubber, synthetic rubber, or a combination thereof. Alternatively, the spring may comprise of materials or mechanisms that are well known to one skilled in the art to have an elastic property that may regain its shape/position after it is stretched or compressed.

When the cutting device is actuated the spring may either be compressed or stretched depending on the particular design of the cutting device. In one variation, one end of the spring is connected to the inside of the housing and the other end couple to the blade. When the cutting device is activated, the spring is stretched and allows the blade to protrude from the housing. When the device is deactivated, the spring contracts and brings the blade back inside the chamber. In another variation, the spring is positioned in such a way that when the device is activated, the spring in compressed. Thus, in this variation when the device is deactivated, the spring expands into its original position pushing the blade back into the chamber.

The cutting device may further comprise an actuating member. The actuating member may be coupled to the spring and the blade. The acutating member may be a knob or piece of material, such as plastic slidably disposed in the chamber. The movement of the actuating member may compress or expand the spring and allow the blade to protrude from the housing. In one variation, the housing has an opening adapted for placement of the actuating member. An actuating member may be partially positioned within the housing and partially protrude from the housing, such that the user may use his finger to move the actuating member and force the blade to exit the housing.

In another variation, markers may be provide on the housing along the sliding path of the actuating member such that the operator may use the marker as guidance to determine the amount of blade extension and hence the amount of incision induced by the blade. In another variation, the spring is comprised of a spring arm. One end of the spring arm may be connected to the blade and the other end connected to a wall inside the housing. When the spring arm is compressed, whether through direct pressure from users finger or through an actuating member, the spring expends sideways and forces the blade to protrude from the housing. In an alternative design, the spring and/or the actuating member may be slidably disposed within a spring ramp. The spring ramp may allow the spring to expend and compress without interference from other mechanical parts. The spring ramp may be an independent channel separate from the guidewire channel. Alternatively, the spring may share the same channel as the guidewire.

The housing may be comprised of various metals, metal alloys, ceramics, rubber, plastic, polymers or combination thereof. Examples of polymers that may be utilized for the fabrication of the housing include, but not limited to, Polycarbonate, Polypropylene, Acrylonitrile Butadiene Styrene (ABS), Polyvinylchloride (PVC), and Polysulfone. The housing may also be constructed of various polymers or malleable plastic that one skilled in the art would consider suitable for constructing a surgical instrument. One may also design the housing structure and the select fabrication material to minimizes cost of production. For example, in one variation, all the components of the cutting device, except the blade and the spring, may be fabricated from molded plastic such as Acrylonitrile Butadiene Styrene (ABS). The housing may be easily molded of plastic material. In one example, the housing is comprised of two mating halves. In such a design, after the remaining components are assembled onto the front housing half, the rear housing half may be snapped into engagement with front housing half.

The economy of materials and assembly may permit marketing the device as a completely disposable device. Alternatively, the cutting device may be designed for repeated use, where after each use the device may be opened up and the blade may be replaced. The cutting device may be sterilized before the next use. The cutting device may have a low profile such that it may be dispose into a conventional biological waste disposal container. The low profile may make it easy for the medical practitioner to insert the used cutting device through the slot on the waste disposal container. The width of the cutting device is preferably less than 3 centimeters, more preferably less than 2 centimeters, and most preferably less than 1.5 centimeters.

In another aspect of the invention, the housing is comprised of transparent or translucent materials. The housing may also comprise of a combination of transparent and translucent materials. In one variation, the housing is comprised of clear plastic parts such that the operator of the device may see through the cutting device and observe the area around the guidewire entry point while the cutting device is positioned over the site of incision. In alternative variations, the cutting device may include other clear (optically transparent, partially transparent or translucent) materials, such as glass, polymers, plastic, crystal, laminates, or the like for the operator to see through the cutting device and observe actuation of the blade. Having a translucent and/or transparent device may limited obstruction of the view of the site of insertion and/or limit blockage of light at the cite of insertion.

In yet another aspect of the invention, the cutting device may include a locking mechanism for securing the blade. The locking mechanism may be adapted such that when the blade is actuated and extends outside the housing, it may be maintained at the extended position until the locking mechanism is released. Alternatively, the locking mechanism may be adapted such that once the cutting device is used, the operator may active the locking mechanism to prevent accidental actuation of the blade. Locking mechanisms that are well known to one skilled in the art that are suitable for securing a slidable blade may be implemented in this cutting device. In another variation, the locking mechanism may include a breakable plastic part or a irreversible locking mechanism such that once the locking mechanism is activated the blade can not be actuated again and thus must be disposed of.

Another aspect of the invention involves a method for utilizing the cutting device. Once a guidewire is inserted into the body of a patient, the cutting device may be utilized to enlarge the hole at incision site. The guidewire may be inserted into a patient's blood vessel with the Seldinger technique, which is well known to one skilled in the art. The cutting device may then be placed over the guidewire at the distal end of the guidewire and slid along the guidewire until the cutting device comes into contact with the patient's body at the site of incision. The cutting device is actuated to extend the blade out of the housing, thus allowing the blade to make a cut into the skin at the edge of the guidewire to increase the hole at the site of the incision. The actuating member may be released to allow the blade to retract back into the housing. The cutting device may then be removed and disposed of. A catheter may be inserted over the guidewire and into the patient's body through the enlarged hole at the point of incision. As discussed above, the cutting device may be placed onto the guidewire laterally, if the channel has a lateral opening. Furthermore, the blade in the cutting device may be extended before the cutting device is moved to the incision site. In this case, as the cutting device is slid onto the incision site the blade may penetrate the skin and make a cut on the skin tissue. It may also be possible to extend the blade before placement of the cutting device over the guidewire.

The present invention may provide various advantages over conventional methods for enlarging the entry hole around the guidewire. The cutting device disclosed in this application may provide improved precision and simplify the incision procedure such that nurses and clinicians are able to enlarge the hold at the incision site with less time and effort. In addition, since the blade may be retraced automatically after the release of the actuating member at the end of the procedure, accidental injuries caused by exposed blades may be minimized. Furthermore, the present cutting device may be designed in such a way that it may be implemented with one hand, and the low profile design may allow easy disposal of the device in a sharp container.

### BRIEF DESCRIPTION OF THE DRAWINGS

In the accompanying drawings, reference characters refer to the same parts through out the different views. The drawings are intended for illustrating some of the principles of the cutting device and are not intended to limit the description in any way. The drawings are not necessarily to scale, emphasis instead being placed upon illustrating the depicted principles in a clear manner.

FIG. 1 is a cross-sectional view of one variation of a cutting device, shown with the blade in the retracted position.

FIG. 2 is a cross-sectional view of the same device illustrated in FIG. 1 with its blade actuated and partially extended outside the housing.

FIG. 3 illustrates another variation of the cutting device where the spring is compressed when the blade is actuated. For illustration purposes, the front housing half and the rear housing half are shown in a partially detached position with a small gap between the two halves.

FIG. 4 illustrates the same device in FIG. 3 with the front housing half and the rear housing have completely detached. The front housing half is shown on the right and the rear housing half is shown on the left.

FIG. 5 illustrates a variation of the cutting device with indentation pattern on the right lateral side of the housing.

FIG. 6 illustrates another variation of the cutting device with a lateral opening along the length of the channel for placement of a guidewire through the rear housing half of the cutting device.

FIG. 7 illustrates yet another variation of the cutting device with a build-in mechanism for adjusting the guidewire insertion angle.

FIG. 8 shows a schematic block diagram of one variation of method for utilizing the cutting device.

### DETAILED DESCRIPTION OF THE INVENTION

The following detailed description should be read with reference to the drawings, in which like elements in different drawings are identically numbered. The drawings, which are not necessarily to scale, depict selected embodiments and are not intended to limit the scope of the invention.

Those skilled in the art will recognize that many of the examples of preferred embodiments provided have suitable alternatives which may be utilized. Thus, it is to be understood that unless otherwise indicated this invention is not limited to specific blade, spring, polymer, or the like, as such may vary. It is also to be understood that the terminology used herein is for the purpose of describing particular aspects of the invention only, and is not intended to be limiting.

A catheter and a guidewire are used herein as an example application to illustrate the functionality of the different aspects of the invention disclosed herein. It will be understood that embodiments of the present invention may be applied in a variety of processes and are not limited to introducing a catheter into a mammalian vessel. Variations of the present invention may be adapted for introducing other percutaneous devices or conduits into a mammalian body. It will also be understood that embodiments of the present invention may be applied over other introducer devices, and it is not limited to applications with a guidewire.

It must also be noted that, as used in this specification and the appended claims, the singular forms “a,”“an” and “the” include plural references unless the context clearly dictates otherwise. Thus, for example, the term “a spring” is intended to mean a single spring or a combination of springs, “a blade” is intended to mean one or more blades, and the like.

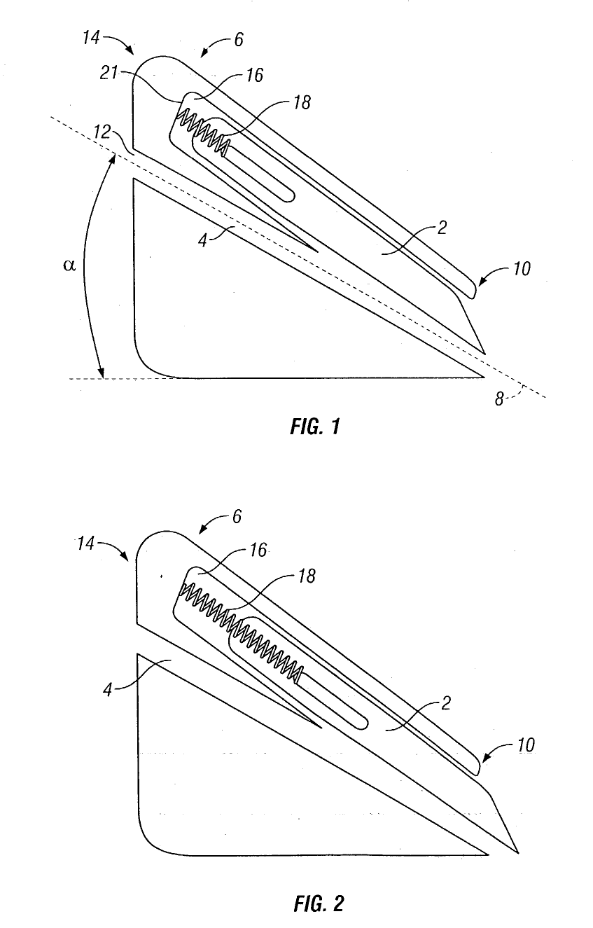

Referring now to FIG. 1, a cutting device having a retractable blade 2 and a channel 4 for positioning of the device around a guidewire is illustrated. In this embodiment of the cutting device, the housing 6 is wedge-shaped and provides the guidewire channel 4 such that the insertion of a guidewire (not shown) through the guidewire channel 4 will result in the guidewire exiting the channel at a predefined incision angle α. In this example, α=30 degrees. The guidewire channel 4 in this variation is enclosed within the housing 6 and runs from the distal opening 8 on the distal end 10 of the cutting device to the proximal opening 12 on the proximal end 14 of the cutting device. The cutting device may be placed over the guidewire by inserting the guidewire into one of the openings 8, 12 of the guidewire channel 4 and sliding the device over the guidewire.

In a blade channel 16, which may be partially connected to the guidewire channel 4, sits a surgical blade 2. The surgical blade 2 may be a No. 11 scalpel blade. No. 11 scalpel blades are commonly used by most nurses and clinicians so that it may be easier for them determine the amount of incisions needed to accommodate various sizes of splittable sheath/dilators. A spring 18 may be placed in the blade channel 16 with one end of the spring 18 connected to an inner surface 21 of the housing and the other end coupled to the blade 2. The spring 18 may be directly connected to the blade 2, or alternatively, the spring 18 may be connected to a secondary member that is connected to the blade 2.

In one embodiment of the invention, the blade is connected to an actuating member which is connected to the spring 18. The actuating member transfers pressure delivered by the user to the blade 2 in order to force the blade 2 out of the housing. The actuating member 20 may be a plastic part that is connected to the blade 2 and partially protrude from the housing 6, thus allowing the user's figure to come into contact with actuating member 20. The actuating member may comprise one or more parts. The housing may have a slot allowing the actuating member to move within it in a predefined path. Although a wedge-shaped housing is shown here, it is understood that the housing may assume various shapes and geometries (e.g, cube-shape, pentagon-shape). The surface of the housing may also have curvatures.

When pressure is applied to the blade 2, the spring 18 may expand and the blade 2 may be displaced and protrude from the housing 6, as seen in FIG. 2. Once the pressure is released the spring 18 may contract and the blade 2 may be pulled back into the housing 6 of the cutting device. In an alternative design the blade channel 16 may be independent of the guidewire channel 4 and exit the cutting device at its own independent channel opening. A spring ramp may be provided for supporting the spring as it expands and compresses within the housing.

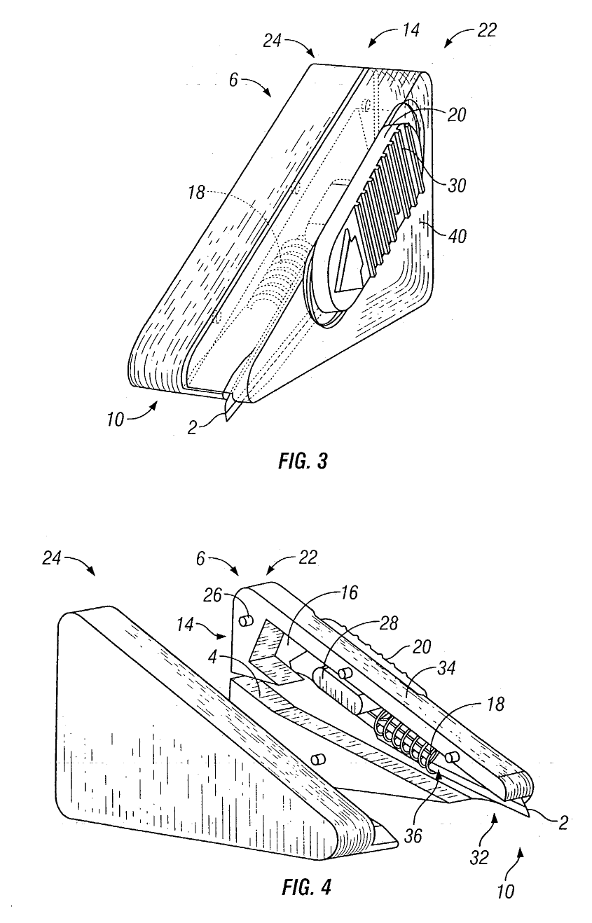

In another embodiment, the cutting device is comprised of two plastic parts that form a small channel for over the wire placement, as shown in FIG. 3. In this particular example, the moving parts (e.g., blade 2, actuating member 20, spring 18) may be assembled into the front housing unit 22 initially. As shown in FIG. 4, the rear housing unit 24 may then be snapped onto the front housing unit by aligning the locking members 26 on the front housing unit 22 with its corresponding receiving holes (not shown) on the rear housing unit 24. The spring 18 may be positioned within a blade channel 16 within the housing. The actuating member 20 may extend within the chamber such that the distal end 28 of the actuating member 20 is positioned behind the spring 18. A blade 2 that is slidably positioned within the blade channel 16 may be connected directly to the actuating member 20.

Alternatively, the blade 2 may be coupled to the actuating member 20 through one or more connecting members such that movement of the actuating member 20 may induce the movement of the blade 2. When pressure is applied on the contact surface 30 of the actuating member 20, and the actuating member 20 is slid forward toward the distal end 10 of the cutting device, the distal end 28 of the actuating member 20 forces the spring 18 to compress and the blade 2 to extend outward through the opening 32 on the distal end 10 of the cutting device. In one variation, the spring l 8 is not directly attached to the housing 6 or the actuating member 20, as seen in FIG. 4. When the cutting device is not actuated, the spring 18 sits in a slot within the housing in an relaxed and unbiased condition. When the device is actuated, the actuating member 20 maintains contact with the proximal end 34 of the spring and applies pressure to the spring 18. The distal end 36 of the spring maintains contact with at least one surface in the housing and forces the spring to compress. When the actuating pressure is released, the spring 18 expands and forces the actuating member 20 to move towards the proximal end 14 of the housing, and as a result brings the blade 2 back into the housing 6.

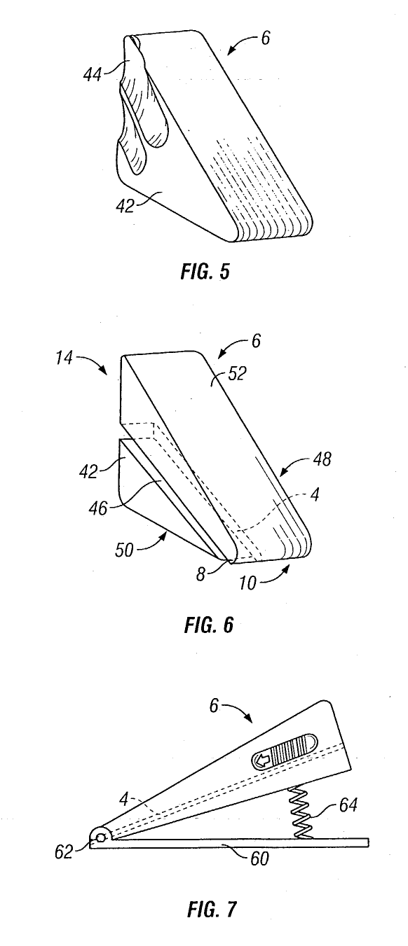

In an alternative embodiment, the spring 18 may be physically connected to the housing 6 and/or connected to the actuating member 20. In one variation of this embodiment, markers 40 are provided on the surface of the housing, such that from the position of the actuating member 20 relative to the markers 40, the operator may determine the size of incision made by the blade 2. In yet another variation, the lateral surface 42 on the rear housing (i.e., the right side surface of the device) may have groves or patterns 44 assisting the operator of the device to position his or her finger on the device, as seen in FIG. 5.

In another aspect of the invention, a lateral opening 46 is provided such that access is allow along the length of the channel 4, as illustrated in FIG. 6. The lateral opening 46 allows the guidewire to be placed into the channel 4 without the need to thread the guidewire through the opening 8 of the channel at the distal end 10 of the housing. Once the cutting device with the lateral opening 46 is positioned on the device, the operator may slide the cutting device along the length of the guidewire toward the location of the incision.

A flexible flap or flaps (e.g. rubber flap or silicon flap) may be positioned at the lateral openings 46 on the cutting device such that placement of the cutting device over the guidewire through the lateral opening 46 requires the user to overcome the resistance caused by the flap. The rubber flap may prevent the guidewire from prematurely slipping out of the channel 4 while operating the device. The flap or other force resistive material may also be place inside the channel 4. Although in FIG. 6 the lateral opening is shown on the right side surface 42 of the device, it is within the contemplation of this invention that the lateral opening 46 may be located on the left side surface 48, the bottom surface 50, or the top surface 52 of the housing 6 depending on the particular design criteria. The position of the mechanical parts may be adjusted in the design to accommodate the location of the channel 4 and/or the lateral opening 46, as one skilled in the art will appreciate.

Referring to FIG. 7; another aspect of the cutting device with an adjustable base 60 is illustrated. A pivot 62 may be provided such that the housing 6 of the cutting device may pivot along a base 60 that is connected to the housing through the pivot 62. This may allow the guidewire channel angle be adjusted relative to the base 60 of the cutting unit. A locking mechanism 64 may be provided to secure the position of the insertion angle.

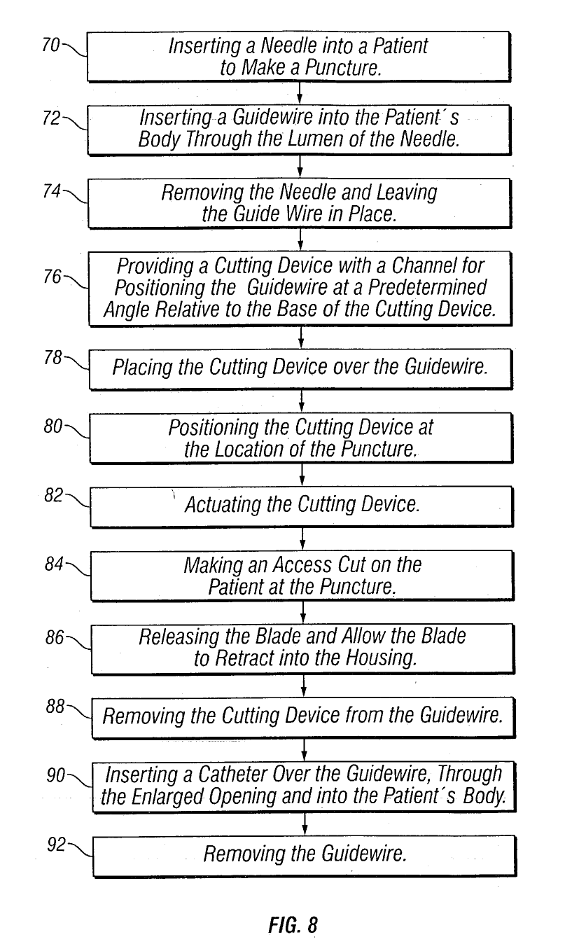

This invention also includes various methods for utilizing the disclosed cutting device, one of which is illustrated in the schematic block diagram of FIG. 8. The initial step 70 comprises inserting a needle into a patient to make a puncture (at this point the distal end of the needle may be positioned inside a targeted vessel). In a next step 72, a guidewire is inserted into the patients body through the lumen of the needle and a portion of the guidewire length may be advanced into the vessel. In step 74, the needle is removed, leaving the guidewire in place. Step 76 comprises providing a cutting device with a channel for positioning the guidewire at a predetermined angle relative to the base of the cutting device. In step 78, the cutting device is placed over the guidewire, which may include inserting the guidewire into the distal end of the guidewire channel and sliding the cutting device toward the puncture location where the guidewire enters the body.

In step 80, the cutting device is positioned at the location of the puncture, including potentially sliding the cutting device along the guidewire until the Cutting device comes into contact with the patient's body. In step 82, the cutting device is actuated, which may include the step of applying pressure on an actuating member and extending the blade out of the housing of the-cutting device. In step 84 an access cut is made on the patient at the puncture location and in step 86, the blade is released and allowed to retract into the housing. In step 88, the cutting device is removed from the guidewire and in step 90, a catheter is inserted over the guidewire through the now enlarged opening and into the patient's body. Finally, in step 92 the guidewire is removed from the patient's body.

All publications and patent applications cited in this specification are herein incorporated by reference in their entirety as if each individual publication or patent application were specifically and individually indicated to be incorporated by reference. This invention has been described and specific examples of the invention have been portrayed. The use of those specifics is not intended to limit the invention in anyway. Additionally, to the extent there are variations of the invention, which are within the spirit of the disclosure or equivalent to the inventions found in the claims, it is our intent that this patent will cover those variations as well.

What is claimed is:

1. A cutting device with a retractable blade for making skin incisions about a guidewire entry location, comprising:
a housing comprising a first opening, a second opening and a channel connecting said first and second openings, wherein a guidewire passing through said channel will exit said channel at said first opening at a pre-defined angle from a bottom surface of said housing;
a spring disposed in said housing in a relaxed condition, wherein said spring is compressed when said device is actuated, said spring having a first end and a second end, wherein said first end of said spring abuts at least one inner surface of said housing when said device is actuated;
an actuating member, wherein at least a portion of said actuating member is slidably disposed in a third opening on said housing, wherein when said device is actuated said actuating member abuts said second end of said spring; and
a blade coupled to said actuating member, said blade being slidably disposed within, said housing, wherein said blade is contained within said housing when said device is not actuated, and wherein said blade protrudes from said housing at or about said first opening when said device is actuated.

2. The cutting device according to claim 1, wherein said predefined angle is between approximately 10 and 45 degrees.

3. The cutting device according to claim 1, wherein said predefined angle is between approximately 25 and 35 degrees.

4. The cutting device according to claim 1, wherein said predefined angle is approximately 30 degrees.

5. The cutting device according to claim 1, wherein said spring is connected to said actuating member at said second end of said spring.

6. The cutting device according to claim 5, wherein said spring is connected to said housing at said first end of said spring.

7. The cutting device according to claim 1, wherein said housing further comprises opposing right and left side surfaces, wherein said third opening is located on said left side surface and said right side surface comprises at least one finger indentation to facilitate handling of said device.

8. The cutting device according to claim 1, wherein said housing comprises an elongated slot, said blade being slidably positioned therein, wherein said elongated slot serves as a constraint on the sliding motion of said blade such that when said device is actuated said blade slides along a pre-determined incision path.

9. The cutting device according to claim 1, wherein at least a portion of said housing is transparent.

10. The cutting device according to claim 1, wherein at least a portion of said housing is translucent.

11. The cutting device according to claim i, wherein said spring comprises a resilient plastic.

12. The cutting device according to claim 1, wherein said spring comprises an elastic metal.

13. The cutting device according to claim 1, wherein said spring comprises an elastic alloy.

14. The cutting device according to claim 1, wherein said spring comprises an elastic polymer.

15. The cutting device according to claim 1, wherein said channel is exposed through at least one surface on said housing to allow a guidewire to be placed therein by passing through said at least one surface.

16. The cutting device according to claim 15, further comprising a cover placed over said exposed channel on said at least one surface to secure said guidewire within said channel.

17. The cutting device according to claim 16, further comprising a locking mechanism connected to said cover for securing said cover to said housing.

18. The cutting device according to claim 1, wherein said housing further comprises opposing right and left side surfaces, wherein said channel is exposed through said right side surface to allow a guidewire to be placed therein by passing through said right side surface.

19. The cutting device according to claim 18, further comprising a locking mechanism to secure a guidewire within said channel.

20. The cutting device according to claim 1, further comprising a locking mechanism for precluding inadvertent actuation of said device.

21. The cutting device according to claim 1, wherein said blade comprises surgical grade stainless steel.

22. The cutting device according to claim 1, wherein said blade comprises a No. 11 scalpel blade.

23. The cutting device according to claim 1, wherein said housing has a spring ramp supporting said spring and upon which said spring slides as said spring compresses and expands.

24. A cutting device with a retractable blade for making skin incisions at a guidewire entry location, comprising:
a housing comprising a first opening, a second opening and a channel connecting said first and second openings, wherein a guidewire passing through said channel will exit said channel at said first opening at a pre-defined angle from a bottom surface of said housing;
a spring disposed in said housing in a relaxed condition, wherein said spring is extended when said device is actuated, said spring having a first end and a second end, wherein said second end of said spring is connected to at least one inner surface of said housing; and
a blade coupled to said first end of said spring, said blade being slidably disposed within said housing, wherein said blade is contained within said housing when said device is not actuated, and wherein said blade protrudes from said housing at or about said first opening when said device is actuated.

25. The cutting device according to claim 24, wherein said predefined angle is between approximately 10 and 45 degrees.

26. The cutting device according to claim 24, wherein said predefined angle is between approximately 25 and 35 degrees.

27. The cutting device according to claim 24, wherein said predefined angle is approximately 30 degrees.

28. The cutting device according to claim 24, further comprising an actuating member coupled to said blade, wherein at least a portion of said actuating member is slidably disposed in a third opening on said housing.

29. The cutting device according to claim 28, wherein said housing further comprises opposing right and left side surfaces, wherein said third opening is located on said left side surface and said right side surface comprises at least one finger indentation to facilitate handling of said device.

30. The cutting device according to claim 24, wherein said housing comprises an elongated slot, said blade being slidably positioned therein, wherein said elongated slot serves as a constraint on the sliding motion of said blade such that when said device is actuated said blade slides along a pre-determined incision path.

31. The cutting device according to claim 24, wherein at least a portion of said housing is transparent.

32. The cutting device according to claim 24, wherein at least a portion of said housing is translucent.

33. The cutting device according to claim 24, wherein said spring comprises a resilient plastic.

34. The cutting device according to claim 24, wherein said spring comprises an elastic metal.

35. The cutting device according to claim 24, wherein said spring comprises an elastic alloy.

36. The cutting device according to claim 24, wherein said spring comprises an elastic polymer.

37. The cutting device according to claim 24, wherein said spring comprises at least one spring arm.

38. The cutting device according to claim 37, further comprising an actuating member, wherein said actuating member is located in a third opening on said chamber, and wherein manually squeezing said actuating member will force said spring arm to bend, thereby advancing said blade outward through said housing.

39. The cutting device according to claim 24, wherein said channel is exposed through at least one surface on said housing to allow a guidewire to be placed therein by passing through said at least one surface.

40. The cutting device according to claim 39, further comprising a cover placed over said exposed channel on said at least one surface to secure said guidewire within said channel.

41. The cutting device according to claim 40, further comprising a locking mechanism connected to said cover for securing said cover to said housing.

42. The cutting device according to claim 24, wherein said housing further comprises opposing right and, left side surfaces, wherein said channel is exposed through said right side surface to allow a guidewire to be placed therein by passing through said right side surface.

43. The cutting device according to claim 42, further comprising a locking mechanism for securing a guidewire within said channel.

44. The cutting device according to claim 24, further comprising a locking mechanism for precluding inadvertent actuation of said device.

45. The cutting device according to claim 24, wherein said blade comprises surgical grade stainless steel.

46. The cutting device according to claim 24, wherein said blade comprises a No. 11 scalpel blade.

47. The cutting device according to claim 24, wherein said housing has a spring ramp supporting said spring and upon which said spring slides as said spring extends and retracts.

48. A method of making a skin incision about a guidewire entry location, comprising:
inserting a distal end of a guidewire into a hollow body organ within a mammalian body while leaving a proximal end of said guidewire outside of said body;
placing a cutting device over said guidewire, wherein said cutting device comprises: a housing comprising a first opening, a second opening and a channel connecting said first and second openings, wherein a guidewire passing .through said channel will exit said channel at said first opening at a pre-defined angle from a bottom surface of said housing; a spring disposed in said housing in a relaxed condition, said spring having a first end and a second end, wherein said first end of said spring abuts at least one inner surface of said housing when said device is actuated; an actuating member, wherein at least a portion of said actuating member is slidably disposed in a third opening on said housing, wherein when said device is actuated said actuating member abuts said second end of said spring; and a blade coupled to said actuating member, said blade being slidably disposed within said housing, wherein said blade is contained within said housing;
actuating said cutting device, wherein said spring is compressed and said blade protrudes from said housing at or about said first opening; and
making an incision about said guidewire entry location.

49. The method according to claim 48, wherein the step of placing a cutting device over said guidewire comprises the steps of:
sliding the cutting device over said guidewire at a proximal end of said guidewire; and
sliding said cutting device along said guidewire until said cutting device comes into contact with said mammalian body.

50. A method of making a skin incision about a guidewire entry location, comprising:
inserting a distal end of a guidewire into a hollow body organ within a mammalian body while leaving a proximal end of said guidewire outside of said body;
placing a cutting device over said guidewire, wherein said cutting device comprises: a housing comprising a first opening, a second opening and a channel connecting said first and second openings, wherein a guidewire passing through said channel will exit said channel at said first opening at a pre-defined angle from a bottom surface of said housing; a spring disposed in said housing in a relaxed condition, said spring having a first end and a second end, wherein said second end of said spring is connected to at least one inner surface of said housing; and a blade coupled to said first end of said spring, said blade being slidably disposed within said housing; wherein said blade is contained within said housing;
actuating said cutting device, wherein said spring is extended and said blade protrudes from said housing at or about said first opening; and
making an incision about said guidewire entry location.

51. The method according to claim 50, wherein the step of placing a cutting device over said guidewire comprises the steps of:
sliding the cutting device over said guidewire at a proximal end of said guidewire; and
sliding said cutting device along said guidewire until said cutting device comes into contact with said mammalian body.

52. A method of making a skin incision about a guidewire entry location, comprising:
inserting a distal end of a guidewire into a hollow body organ within a mammalian body while leaving a proximal end of said guidewire outside of said body;
placing a cutting device over the guidewire at said proximal end of said guidewire, wherein said cutting device comprises a retractable blade, and wherein said guidewire forms an approximately 30 degree angle with a bottom surface of said cutting device;
sliding said cutting device along said guidewire until said cutting device comes into contact with said mammalian body;
actuating said cutting device; and
making an incision about said guidewire entry location.

53. A retractable surgical blade comprising:
a wedge-shaped housing having a bottom surface, said housing further having a channel, said channel providing a straight path from an opening on a first side of said housing to a second opening on said second side of said housing, said straight path and said bottom surface forming an angle of approximately 30 degrees, said housing comprising at least two detachable polymer parts;
a spring disposed in said housing in a relaxed condition, wherein said spring is compressed when said device is actuated, said spring having a first end and a second end, wherein said first end of said spring abuts at least one inner surface of said housing when said device is actuated;
an actuating member, wherein at least a portion of said actuating member is slidably disposed in a third opening on said housing, wherein when said device is actuated said actuating member abuts said second end of said spring; and
a No. 11surgical blade connected to said actuating member, said blade being slidably disposed within said housing, wherein said blade is contained within said housing when said device is not actuated, and wherein said blade protrudes from said housing at or about said first opening when said device is actuated.
